(19) United States
(12) Patent Application Publication
(10) Pub. No.: US 2011/0118712 A1
(43) Pub. Date: May 19, 2011

(54) LASER CORRECTION OF VISION CONDITIONS ON THE NATURAL EYE LENS

(76) Inventors: Holger Lubatschowski, Gehrden (DE); Uwe Oberheide, Koeln (DE); Alfred Wegener, Meckenheim (DE); Silvia Schumacher, Hannover (DE)

(21) Appl. No.: 12/735,450

(22) PCT Filed: Jan. 16, 2009
(86) PCT No.: PCT/EP2009/000274

§ 371 Date: Jan. 24, 2011

(30) Foreign Application Priority Data
Jan. 18, 2008 (DE) 102008005053.9

(51) Int. Cl. A61F 9/008

(52) U.S. Cl. 606/5

(57) ABSTRACT

The invention relates to an ophthalmologic laser system (1) comprising an ultra-short pulse laser (2) for outputting ultra-short laser pulses (3), focusing optics (4) for producing at least one focal point (5) on and/or in the eye lens (6) of the patient's eye (7), a deflection mechanism (9) for varying the position of the focal point (5) on and/or in the eye lens (6), and comprising a control mechanism (11) for controlling the deflection mechanism (9). The laser system (1) is characterized in that the laser pulses output by the ultra-short pulse laser (2) and the size of the focal point (5) fixed by the focusing optics (4) are configured such that a fluence can be applied below or on the disruption threshold of the material of the eye lens (6) at the focal point (5), wherein said fluence is at the same time sufficiently high to cause changes in at least one material property of the material of the eye lens (6). The laser system (1) is also characterized in that the deflection unit (9) can be actuated by means of the control mechanism (11) in such a way that the focal points (5) of a group of laser pulses (3) are arranged such that a diffractive optical structure (20) can be produced by the changes in the material property in the eye lens (6) caused by way of application of the laser pulses. The invention also relates to a method for generating control data for actuating a deflection unit (9) of such a laser system (1).

The present invention relates to a novel laser system and method for correcting vision conditions, such as farsightedness (hyperopia), nearsightedness (myopia), astigmatism, or presbyopia. The laser system and the method according to the invention intend to carry out the correction of the vision condition by treating or processing the natural eye lens of a patient.

Ultra-short laser pulses of a duration within the range of some femtoseconds (fs) to picoseconds (ps) are known to generate disruptions in or on transparent media by means of the so-called optical breakthrough. Disruption leads to a removal or tearing off of material. The interaction process is based on multiphoton absorption and has been already described in a plurality of publications (cf. for example Alfred Vogel and Vasan Venugopalan: “Mechanisms of Pulsed Laser Ablation of Biological Tissues”; Chem. Rev. 2003, 103, 577-644; or U.S. Pat. No. 5,656,186 A or U.S. Pat. No. 5,984,916 A). It is on the one hand characteristic that the disruption generated by the laser is locally very restricted, and on the other hand, that in materials transparent to laser radiation, the site of disruption can be freely placed in three dimensions.

U.S. Pat. No. 6,552,301 B2 extensively deals with the drilling of holes by means of ultra-short laser pulses. It is noted in a side remark that one can also work inside the material. It is further indicated only very briefly and without giving any details that ultra-short laser pulses can also be used for photorefractive surgery.

In ophthalmology, material removal by means of the optical breakthrough is used in the field of refractive surgery, i.e. for interventions and operations for correcting the refractive power of the eye. DE 199 38 203 A1 and DE 100 24 080 A1, of which the contents are nearly identical, in quite general words describe several different methods, in particular the reshaping of the cornea by material removal by means of pulsed lasers, among others by ultra-short pulse laser.

DE 10 2004 033 819 A1 also describes, among other things, methods of refractive surgery with fs pulses. For treating presbyopia, WO 2005/070358 A1 suggests to make cuts in the surface of the natural eye lens through material removal by means of fs laser pulses to increase the elasticity of the eye lens and thus its power of accommodation.

Further examinations on the consequences of photodisruption in refractive surgery of the cornea of the eye can be found in Kurtz R M, Horvath C, Liu H H, et al.: “Lamellar refractive surgery with scanned intrastromal picosecond and femtosecond laser pulses in animal eyes”, J Refract Surg. 1998; 14:541-548; or in R. Krueger, J. Kuszak, H. Lubatschowski et al.: “First safety study of femtosecond laser photodisruption in animal lenses: Tissue morphology and cataractogenesis”, Journal of Cataract & Refractive Surgery, Volume 31, Issue 12, Pages 2386-2394. Here, it showed in the cornea of the eye that changes which are caused within the corneal stroma with moderate laser energy, for example for cutting a so-called corneal flap for the LASIK operation, completely heal up within only a few days to weeks and do not leave any visible changes [Heisterkamp A, Thanongsak M, Kermani O, Drommer W, Welling H, Ertmer W, Lubatschowski H: “Intrastromal refractive surgery with ultrashort laser pulses—in vivo study an rabbit eyes”; Graefes Archives of Clinical and Experimental Ophthalmology 241(6), 511-517 (2003)]. At least, the penetrating light is not influenced to such an extent that the treated patients are disturbed by it.

The lower the pulse energy used, and the higher the focusing (i.e. the higher the numerical aperture, NA, of the focusing optics), the more precise, i.e. smaller as to its dimensions, is the laser-induced disruption and the thus achieved material removal. However, the optical breakthrough is a threshold process. Depending on the material of the workpiece, there is a threshold also referred to as “removal threshold” or “disruption threshold” (indicated in intensity or energy over area, i.e. fluence), below which no disruption nor material removal occurs.

However, even below the disruption threshold, a change in the material properties of the workpiece can still occur. It can be a chemical change caused by free electrons that have been formed by multiphoton absorption or comparable, laser-induced ionization processes. It can also be photochemical changes that have been, for example, caused by non-linear generation of blue or UV light. Only with higher energies, photothermally induced or plasma-induced local fractures of the medium occur. The change in material properties can be e.g. a locally defined fusion, so that the material contracts locally. Moreover, a locally defined change of the index of refraction and/or the transparency of the material is possible.

This effect below the disruption threshold of the material is already often used, for example for producing light guides in glass [“Micromachining bulk glass by use of femtosecond laser pulses with nanojoule energy”, Chris B. Schaffer, André Brodeur, José F. Garcia, and Eric Mazur, Optics Letters, Vol. 26, Issue 2, pp. 93-95], for writing 3D sculptures in glass, or for changing the index of refraction in plastic material of artificial eye lenses (cf. DE 10 2004 033 819 A1). However, the results of the examinations on natural components of the eye, in particular the cornea, obtained by now, confirmed that the irradiation of laser pulses with fluences on or below the disruption threshold did not result in any changes of the visual faculty of the patient at least in the medium or long term.

Unfortunately, the known methods of refractive surgery still suffer in too many cases on the hand from a lack of predictability of the result, on the other hand from a wound healing process involving complications.

It was the object of the present invention to provide a laser system and a method for correcting vision conditions representing an advantageous alternative to the conventional correction possibilities that can be in particular carried out more quickly.

This object is achieved by a laser system having the features of claim 1 and by a method having the features of claim 17, respectively. Advantageous further developments of the invention are stated in the sub-claims.

The laser system according to the invention is characterized in that the laser pulses output from the ultra-short pulse laser, and the size of the focal point (focus) fixed by the focusing optics are configured (i.e. adjusted with respect to each other) such that a fluence on or below the disruption threshold of the material of the eye lens can be applied at the focal point, this fluence being at the same time sufficiently high to cause changes in a material property of the material of the eye lens. The invention is based on the finding that by the application of ultra-short laser pulses at or below the disruption threshold, permanent material changes can be achieved in the eye lens, for example local changes of the index of refraction and/or transparency. This finding is surprising against the background of the examinations up to now as in the similarly transparent cornea, at least no permanent material changes have been possible. (A possible explanation would be a different wound healing behavior of the cornea and the eye lens, but no more detailed examinations have yet been conducted concerning these backgrounds of the invention.) The fact that by processing the eye lens, vision conditions can be corrected, was not obvious also because the eye lens, compared to the cornea, has a much lower influence on the total refractive power of the eye.

The configuration or adjustment of the laser pulses and the focusing optics in the invention is to be understood as follows: The larger the angle (i.e. the numerical aperture of the focusing optics) at which the laser pulse is focused, the lower the energy of one individual pulse can be at a constant pulse duration, and the more precise the processing of the eye lens is without the removal threshold of the material being exceeded.

In contrast, the shorter the laser pulses with the same numerical aperture of the focusing optics, the smaller may be the pulse energy in order not to exceed the removal threshold of the material. The smaller pulse energy in turn leads to the material changes remaining restricted to a very small volume at the focal point.

The interaction of the pulses of the laser system according to the invention with the material of the eye lens generates tiny lesions. Small changes (without material removal) remain at the site of the interaction. Depending on the selection of the system parameters, they can have dimensions of 1-2 micrometers or even less than one micrometer, for example of one or two tenths micrometers. The interaction can be effected by selecting the position of the focal point in the nucleus of the eye lens as well as in or on the lenticular cortex. The fluence required for interaction at one site does not have to be deposited with one single laser pulse but can rather be introduced into the material by radiating the same site with a plurality of laser pulses.

The laser system according to the invention permits a unique new method for correcting vision conditions. In contrast to conventional methods, it avoids material removal—whereby the formation of wounds at the eye and any possible complications of the wound healing process are avoided at the same time. Compared to the usual methods of refractive surgery, another advantage is that not the cornea, but the eye lens is processed with the method. As the incident light is already bundled by the cornea, smaller structures are sufficient in the eye lens—compared to the cornea—to influence the light. The smaller the required structures, the faster they can be generated—and the less the inconveniences for the patient are.

Particular advantages result by the deflection mechanism being configured to set the focal points of a group of laser pulses such that by the application of the laser pulses in the eye lens, a diffractive, i.e. light diffracting, optical structure can be generated. The lesions can be designed, depending on the selection of the laser parameters, such that incident light is diffracted or dispersed at the points with changed material properties. If a plurality of such lesions is generated, one can, according to the principle of diffractive optics, create image-forming properties within the lens. By means of these image-forming properties, vision conditions of the eyes can be corrected. For example, by generating a focusing effect, the refractive power of the lens can be increased and shortsightedness thus corrected. Or by generating a defocusing effect, the refractive power of the lens can be reduced and farsightedness thus corrected. Moreover, by introducing a cylindrical effect, astigmatism can be corrected. Moreover, by introducing a bifocal effect, the accommodation of the eye could be simulated and presbyopia could thus be corrected.

The diffractive optical structure in the eye lens could be a two-dimensional diffractive structure which would be, compared to other structures, relatively easy to manufacture. The lesions could be placed in one or several, in each case contiguous, “carpets” in the eye lens.

The two-dimensional diffractive structure could in particular comprise a plurality of rings or ellipses concentric with respect to each other which together change the refractive power of the eye lens by corresponding light diffraction. Ellipses offer the possibility of achieving different effects of refractive power in different directions in space and thus e.g. of correcting an astigmatism of the eye.

As an alternative, the diffractive optical structure in the eye lens could be a holographic, i.e. three-dimensional, diffractive structure. This possibility offers itself as the eye lens already provides a three-dimensional medium for accommodating the holographic structure.

Preferably, the control mechanism of the laser system is adapted to actuate the deflection mechanism, taking into consideration the optical influence of the transparent components of the patient's eye on the laser pulses, in particular taking into consideration the optical influence of the cornea of the eye and the front face of the eye lens. This can be realized by detecting a digital image of the optical system of the eye in the laser system, or by entering the same into the laser system, which is then consulted for simulating the result of the treatment and/or for generating control data.

It is moreover advantageous for the control mechanism to be adapted to actuate the deflection mechanism, taking into consideration the optical influence on a laser pulse resulting from the material changes in the eye lens by the preceding laser pulses. For example, the laser pulses could lead to the material of the eye lens locally extending or contracting. This change of the shape of the eye lens should then be taken into consideration in the positioning of the subsequent laser pulses.

Ideally, the focusing optics comprises a numerical aperture (NA) within a range of 0.1 to 1.4, preferably within a range of 0.1 to 0.3. With this comparably strong focusing, very precise and locally restricted lesions or material changes can be generated.

Preferably, the focal point of the focusing optics in the eye lens has a diameter within a range of 0.1 to 10 micrometers, preferably within a range of 0.2 to 3.0 micrometers. In this manner, diffractive structures with a precisely defined geometry can be generated in the eye lens.

The laser pulses of the laser system should have a wavelength within a range of 400-1400 nm, preferably within a range of 700 to 1100 nm, to keep the dispersion and absorption in front of the eye lens (e.g. in the cornea) as low as possible.

Laser pulses having a pulse duration within a range of 10 fs to 1 ps, preferably within a range of 100-500 fs, are particularly advantageous. With these, high-precision lesions can be generated.

Suited pulse energies are within a range of 1 nJ (nanojoule) to 10 μJ (microjoule), preferably within a range of 100 nJ to 3 μJ.

If the laser pulses have a pulse repetition rate within a range of 1 kHz-100 MHz, preferably within a range of 10-1000 kHz, a plurality of lesions can be set within a short time, so that the treatment can be performed quickly and involves a minimum of inconveniences for the patient.

In the laser system, an actuated shutter element for fixing the pulse repetition rate and/or the number of output laser pulses can be provided. Particularly fast response times can be achieved by an acousto-optical modulator or an electro-optical modulator. However, an actuated shutter would also be conceivable.

With the laser system according to the invention, it should be ideally possible to generate, with a laser pulse at the focal point, a fluence within a range of 1×10⁻³ J/cm² to 3.5×10⁴ J/cm², preferably within a range of 0.5 J/cm² to 100 J/cm². These values proved to be particularly advantageous for a sub-disruptive processing of the eye lens material.

To be able to focus the laser pulses precisely to the predetermined sites, a fixing means for fixing the position of the patient's eye relative to the laser system is preferably provided. The positioning of the eye will become particularly stable with a suction ring. As an alternative, a so-called “eye tracker” could be employed if it ensures sufficient precision and a sufficient reaction rate.

The invention also relates to a method for generating control data for actuating a deflection mechanism of an ophthalmologic laser system generating ultra-short laser pulses, which can preferably be one of the above-described variants of a laser system. The control data comprise a group of position control data records, where the deflection mechanism can be actuated by means of one single position control data record, such that a focusing means and the deflection mechanism determine, depending on the position control data record, the three-dimensional position of an optical focal point of laser pulses of the laser system in or on the eye lens of a patient's eye. The group of position control data records is selected such that a diffractive or holographic structure can be generated in the eye lens of a patients' eye if a fluence below the removal threshold of the material of the eye lens is applied at each focal point by means of at least one ultra-short laser pulse.

The control data could be generated in the laser system itself or made available to the laser system wirelessly or wire-bound, or via a suited interface in the form of a file or a data stream.

It is advantageous for the position control data to fix the sequence of a plurality of focal points consecutively generated at different sites. This sequence could then be selected such that the lesions generated by preceding laser pulses do not have any effect on subsequent pulses, or that adjacent lesions are not generated directly one after another, so that the material of the eye lens has more time to relax upon the laser's influence.

Each position control data record could comprise two-dimensional coordinates of a focal point if the position of the focal points is invariably fixed by the focusing optics in the z-direction, i.e. in the direction of the optical axis of the eye. Otherwise, a position control data record could also comprise three-dimensional coordinates. The z-coordinate would then be employed for actuating the focusing means. The position control data could be represented as Cartesian coordinates or as cylindrical coordinates.

Preferably, the control data are adapted to actuate the focusing means and/or the deflection mechanism, taking into consideration the optical influence of the transparent components of the patient's eye on the laser pulses, in particular taking into consideration the optical influence of the cornea of the eye and the front face of the eye lens, to be able to place the focal points precisely at the desired sites. To this end, a standard model of an eye could be used. However, it is better to consider a digital, three-dimensional, individual model of the eye to be treated. This digital model can be in turn obtained, adjusted to the patient, by imaging methods, such as Optical Coherence Tomography (OCT) or ultrasonic imaging, before or during the intervention. If the laser system has an imaging means, this could be consequently act as real-time supervision of the processing results during the treatment.

As already illustrated, the control data could also be adapted to actuate the deflection mechanism, taking into consideration the optical influence on a laser pulse resulting from the changes in the material or shape of the eye lens by the preceding laser pulses.

Advantageously, the control data comprise synchronization control data for synchronizing the actuation of the deflection mechanism with the output of laser pulses from an ultra-short laser, so that the output of the laser pulses and the respective positioning of the focal points can be ideally adjusted with respect to each other.

The method will become particularly simple and is nevertheless well suited for correcting vision conditions if the group of position control data is selected such that the diffractive structure that can be generated by the application of the laser pulses is two-dimensional and comprises a plurality of rings or ellipses concentric with respect to each other. The structure of concentric rings here serves to uniformly change the refractive power of the eye lens, while astigmatism could be corrected with the elliptic structure.

The diffractive structures should have dimensions in the order of the wavelength of visible light, i.e. in the order of about 0.4 to 1 μm, to be able to influence the incident light by diffraction. Three-dimensional structures and structures other than rings or ellipses would be conceivable.

The position control data could be selected such that the diffractive structure that can be generated by the application of the laser pulses are arranged on a plane or an arched surface.

In most case, it will be advantageous to select the position control data such that the diffractive structure that can be generated by the application of the laser pulses is centered with respect to the optical axis of the patient's eye.

The invention is also reflected in a computer program with a program code for carrying out one of the above-described method variants if the computer program is run on a computer.

The invention is moreover reflected in a refractive-surgical method for treating a patient's eye, wherein a plurality of ultra-short laser pulses are focused on and/or in the natural eye lens of the patient's eye at several different focal points, where a fluence below the removal threshold of the material of the eye lens is applied at the focal point with a laser pulse, but wherein this fluence is at the same time sufficiently high to cause changes in a material property of the material of the eye lens, and wherein the position of the focal points is selected such that a diffractive optical structure is generated in the eye lens of the patient's eye by the influence of the focused laser pulses.

Apart from the above-described method variants, the diffractive structure could be shaped such that the eye lens has two or more different focal lengths after the treatment, e.g. by various refractive powers in different zones relative to the optical axis. In this manner, one could work against presbyopia, i.e. a restricted accommodation capacity of the eye lens.

Below, one advantageous embodiment of the invention will be illustrated more in detail with reference to a drawing. In detail:

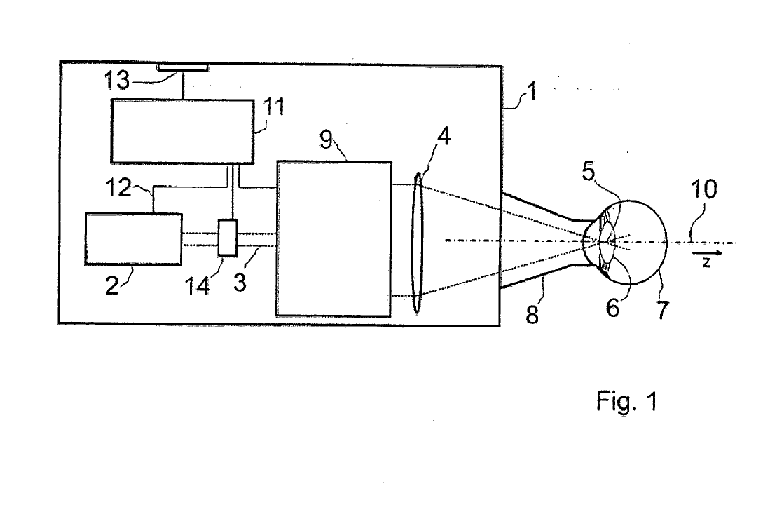

FIG. 1 shows an embodiment of the laser system according to the invention in a schematic representation,

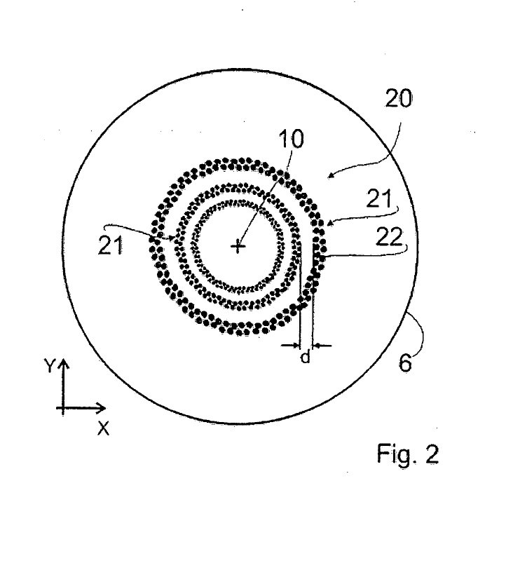

FIG. 2 shows a plan view of an eye lens treated with the method according to the invention along the optical axis of the eye.

FIG. 1 shows, in a schematic representation, an embodiment of a laser system 1 according to the invention. The laser system 1 is in particular an ophthalmologic laser system, i.e. a laser system 1 suited for eye operations. It comprises a laser 2 which outputs laser radiation in the form of ultra-short laser pulses 3. In the preferred embodiment, it is a femtosecond laser 2 with pulse durations within a range of some femtoseconds (fs) to some 100 fs. For minimum maintenance requirements, e.g. fiber oscillators are preferred, with or without subsequent amplification of the pulses. Typical values for the laser pulses 3 are a pulse duration of 100 fs, a pulse energy of 1 pJ, a wavelength of 700 to 1100 nm, and a repetition rate of 100 kHz.

A focusing optics 4 with a numerical aperture within a range of between 0.1 and 1.4, for example a single lens or a lens system, focuses the laser pulses 3 onto a focal point 5. The focal length of the focusing optics 4 is selected such that the focal point is within or on the eye lens 6 of a patient's eye 7 which is brought into a predefined position that is immovable relative to the laser system 1 during the treatment. As fixing means 8, a suction ring that holds the eye can be used for example. Optionally, instead of the fixing means, an electronic automatic tracking of the laser beam can be used (a so-called “eye tracker”). The electronic tracking detects the movement of the eye, for example by video monitoring, and tracks the movement of the eye 7 with the laser focal point 5 by means of the deflection mechanism 9 and the focusing optics 4.

The focal point 5 preferably has a diameter of only 0.2 to 1 μm, but it can also be somewhat larger. The numerical aperture of the focusing optics 4 and the parameters of the ultra-short laser pulses 3 are adjusted with respect to each other such that a fluence on or below the disruption threshold of the material of the eye lens 6 can be generated at the focal point 5, i.e. for example 5J/cm².

In front of or behind the focusing optics 4, an actuated deflection mechanism 9 is provided in the beam path of the laser 2. A scanner system is suited as deflection mechanism 9, which usually comprises two swiveling mirrors (not shown) with swiveling axes perpendicular with respect to each other. The laser beam 3 can be laterally deflected by means of the swiveling motion of the scanner mirrors. By means of the deflection mechanism 9, the position of the focal point 5 of the laser pulses 3 can be changed two-dimensionally, so that the focal point 5 can be placed at any arbitrary point on a possibly arched surface within the eye lens 6.

The focusing optics 4 can also comprise actuated elements to be able to change the size of the focal point 5 and/or the position of the focal point 5 in the z-direction, i.e. in the direction of the optical axis 10 of the eye 7. In this case, the position of the focal point 5 on or in the eye lens 6 can be varied even three-dimensionally by cooperation of the actuation of the focusing optics 4 and the deflection mechanism 9.

To actuate the laser 2, the focusing optics 4 and the deflection mechanism 9, the laser system 1 comprises a control mechanism 11, for example a programmable microprocessor. The control mechanism 11 generates control data in a format suited for actuating the respective components of the laser system 1. The deflection mechanism 9 requires as control data for example position data records which each determine the position of the two scanner mirrors.

The control mechanism 11 can transmit the control data to all these elements via data lines 12 which connect the control mechanism 11 with the laser 2, with the deflection mechanism 9, and with the focusing optics 4. In this manner, the control mechanism 11 can, for example, take care of a synchronization of the deflection mechanism 9 with the output of the laser pulses 3 by the laser 2 to prevent the deflection mechanism 9 from moving just when the laser pulse 3 is arriving.

The control mechanism 11 comprises an interface 13 via which the patients' data, measured values, command data or other data can be input and subsequently consulted for calculating or generating the control data. The interface 13 can be, for example, a drive, a keyboard, a USB port and/or a wireless interface.

As further optical element, which can also be actuated by the control mechanism 11, a shutter element 14 is provided in the laser system 1. In the embodiment, the shutter element 14 is an acousto-optical or electro-optical modulator, as these modulators have an extremely short response time and can selectively allow or interrupt the laser radiation between two laser pulses 3. By means of the shutter element 14, the number of output laser pulses 3 can be consequently fixed, and moreover, the pulse repetition rate can be optionally reduced.

Hereinafter, the method to be carried out with the ophthalmologic laser system 1 will be described. If no pre-adjusted standard data are used, patients' data are first input into the control mechanism 11 via the interface 13. The patient's data represent the dimensions and/or vision conditions of a patient's eye 7. These can be the results of a preceding measurement of the patient's eye 7.

The control mechanism 11 calculates and generates control data from the available data. These control data are adapted to actuate the focusing means 4 and/or the deflection mechanism 9, taking into consideration the optical influence of the transparent components of the patient's eye 7 on the laser pulses, in particular taking into consideration the optical influence of the cornea of the eye and the front lens face. To this end, the control mechanism 11 could simulate how the vision conditions of the patient change if a certain diffractive optical structure is generated in the eye lens 6 of the patient's eye 7. In this manner, the control mechanism can calculate a diffractive structure ideal for correcting one or several vision conditions of the patient's eye 7. The ideal diffractive structure is selected such that by the diffraction of the incident light at the same, the optical properties of the patient's eye 7 change such that the former vision condition is largely cancelled. For example, the diffractive structure could increase or reduce the refractive power of the patient's eye 7, or it could generate various zones with different refractive powers. From this ideal diffractive structure, one can deduce the positions of the individual fine lesions that must be generated in the eye lens 6 to form the ideal diffractive structure together. The ideal diffractive structure can be two- or three-dimensional.

Based on the above-described calculation, the control data comprise a group of position control data records. The deflection mechanism 9 (and optionally the focusing means 4) is/are actuated by means of one single position control data record, such that the focusing means 4 and the deflection mechanism 9 determine the three-dimensional position of an optical focal point 5 of the laser pulses 3 of the laser system 1 depending on the position control data record. As already illustrated, the group of position control data records is moreover selected such that a diffractive or holographic structure can be generated in the eye lens 6 of a patients' eye 7 if a fluence below the disruption threshold of the material of the eye lens 6 is applied at each focal point 5 by means of at least one ultra-short laser pulse 3. The control data are moreover adapted to actuate the deflection mechanism 9, taking into consideration the optical influence on a laser pulse 3 resulting from the changes in the material or shape of the eye lens 6 by the preceding laser pulses 3.

The eye 7 of a patient to be treated is brought into a defined position relative to the laser system 1 by means of the fixing means 8 and held in this position or tracked, if an automatic tracking (eye tracker) is used. The control data are transmitted from the control mechanism 11 via the data lines 12 to the laser 2, the focusing optics 4; the deflection mechanism 9 and the shutter element 14. A plurality of laser pulses 3 of the laser 2 is output onto the patient's eye 7 and focused in or on the eye lens 6 consecutively at a plurality of focusing points 5. The position of the individual focal points 5 of the laser pulses 3 is fixed by the position control data records and mainly varied by means of the deflection mechanism 9. At each focal point 5, one or several laser pulses 3 are applied. The energy density (fluence) deposited there causes a lesion with a local change of the material properties, preferably a local change of the transparency or the index of refraction. By the plurality of the lesions, altogether a diffractive structure is formed.

A comparatively simple example of such a diffractive structure 20 in the treated eye lens 6 is represented in FIG. 2. FIG. 2 is a view of the patient's eye 7 in the direction of the optical axis 10 of the eye 7. The diffractive structure 20 consists of several rings 21 concentric with respect to each other and to the optical axis 10, three of the rings 21 being represented. Each ring 21 is composed of a plurality of individual adjacent lesions 22 of the eye lens 6 as a contiguous “carpet”, which each have been formed at the site of a focal point 5 of the laser radiation. The site of the individual lesions 22 can be indicated in x-y coordinates to each of which one position control data record corresponds.

The distance d between two rings 21 is in the order of the wavelengths of visible light, but it can also be somewhat larger, i.e. within a range of 0.2 μm to 2.5 μm. The lesions 22 remain in the eye lens 6 permanently (or at least over quite a long period). The diffractive structure 20 can therefore equally permanently correct the vision condition of the treated eye.

In the following table, some parameters are given by way of example which are suited for performing the method according to the invention:

| | | Values for | Typical | Typical |
|---|---|---|---|---|
| | Values for | strong | values | values |
| Parameter | low effect | effect | (Example 1) | (Example 2) |
| Pulse duration T [fs] | 10 | 1000 | 100 | 500 |
| Pulse energy F [nJ] | 1 | 10,000 | 100 | 1000 |
| Mean laser power | 0.1 | 10 | 1 | 2 |
| [MW] | | | | |
| Diameter of focal | 0.2 | 10 | 0.5 | 5 |
| point [μm] | | | | |
| Focal point area A | 3.14E−10 | 7.85E−07 | 1.96E−09 | 1.96E−07 |
| [cm²] | | | | |
| Intensity I [W/cm²] | 1.27E+09 | 3.18E+18 | 5.09E+14 | 1.02E+13 |
| Fluence F [J/cm²] | 1.27E−03 | 3.18E+04 | 5.09E+01 | 5.09E+00 |

The “Values for low effect” take care that the change of the eye lens 6 is as minimal as possible and restricted to a spatially extremely small interaction zone. With these values, the eye lens can be very precisely treated; however, for generating larger surfaces of the diffractive structure 20, possibly too many lesions are required, meaning a correspondingly long duration of treatment. The “Values for strong effect” take care of a large-volume material change. Correspondingly less laser pulses are required for a treatment; however, the material of the eye lens 6 is relatively strongly stressed with the given values. Typical values which are particularly suited for the method are given as “Example 1” and “Example 2”.

Starting from the described embodiments, the laser system and the method according to the invention can be modified in many respects. As mentioned, the diffractive structure 20 can also be a three-dimensional, i.e. holographic structure. It would also be conceivable not to generate a diffractive, but a refractive structure inside the eye lens 6, i.e. a “lens region” with a concave or convex interface and with a higher or lower refractive power than the natural eye lens material.

1. Ophthalmologic laser system (1), having
an ultra-short pulse laser (2) for outputting ultra-short laser pulses (3),
a focusing optics (4) for generating at least one focal point (5) on and/or within the eye lens (6) of a patient's eye (7),
a deflection mechanism (9) for varying the position of the focal point (5) on and/or within the eye lens (6), and
a control mechanism (11) for controlling the deflection mechanism (9),
characterized in that
the laser pulses (3) output by the ultra-short pulse laser (2) and the size of the focal point (5) determined by the focusing optics (4) are configured such that a fluence below or at the disruption threshold of the material of the eye lens (6) can be applied at the focal point (5), said fluence being at the same time sufficiently high to cause changes in at least one material property of the material of the eye lens (6),
and in that the deflection mechanism (9) can be actuated by the control mechanism (11) such that the focal points (5) of a group of laser pulses (3) are arranged such that by the changes in the material property in the eye lens (6) caused by the application of the laser pulses (3), a diffractive optical structure (20) can be generated.

2. Laser system according to claim 1, characterized in that the diffractive optical structure (20) in the eye lens (6) is a two-dimensional diffractive structure.

3. Laser system according to claim 2, characterized in that the two-dimensional diffractive structure (20) comprises a plurality of rings (21) or ellipses concentric with respect to each other.

4. Laser system according to Claim 1, characterized in that the diffractive optical structure in the eye lens (6) is a holographic, three-dimensional diffractive structure.

5. Laser system according to claim 1, characterized in that the control mechanism (11) is adapted to actuate the deflection mechanism (9), taking into consideration the optical influence of the transparent components of the patient's eye (7) on the laser pulses (3), in particular taking into consideration the optical influence of the cornea of the eye (7) and the front surface of the eye lens (6).

6. Laser system according to claim 1, characterized in that the control mechanism (11) is adapted to actuate the deflection mechanism (9), taking into consideration the optical influence on a laser pulse (3) resulting from the material changes in the eye lens (6) by the preceding laser pulses (3).

7. Laser system according to claim 1, characterized in that the focusing optics (4) has a numerical aperture within a range of 0.1 to 1.4, preferably within a range of 0.1 to 0.3.

8. Laser system according to claim 1, characterized in that the focal point (5) of the focusing optics (4) in the eye lens (6) has a diameter within a range of 0.1 to 10 micrometers, preferably within a range of 0.2 to 3.0 micrometers.

9. Laser system according to claim 1, characterized in that the laser pulses (3) have a wavelength within a range of 400 nm to 1400 nm, preferably within a range of 700 nm to 1100 nm.

10. Laser system according to claim 1, characterized in that the laser pulses (3) have a pulse duration within a range of 10 fs to 1 ps, preferably within a range of 100 to 500 fs.

11. Laser system according to claim 1, characterized in that the laser pulses (3) have a pulse energy within a range of 1 nJ to 10 μJ, preferably within a range of 100 nJ to 3 μJ.

12. Laser system according to claim 1, characterized in that the laser pulses (3) have a pulse repetition rate within a range of 1 kHz to 100 MHz, preferably within a range of 10 to 1000 kHz.

13. Laser system according to claim 1, characterized in that an actuated shutter element (14) is provided for determining the pulse repetition rate and/or the number of output laser pulses (3).

14. Laser system according to claim 13, characterized in that the shutter element (14) is an acousto-optical modulator, an electro-optical modulator, or a shutter.

15. Laser system according to claim 1, characterized in that a fluence within a range of 1×10⁻³ J/cm² to 3.5×10⁴ J/cm², preferably within a range of 0.5 J/cm² to 100 J/cm², can be generated at the focal point (5) with a laser pulse (3).

16. Laser system according to claim 1, characterized in that a fixing means (8) for fixing the position of the patient's eye (7) relative to the laser system (1), or an automatic tracking system for the laser beam which considers the eye movement, is provided.

17. Method for generating control data for actuating a deflection mechanism (9) of an ultra-short laser pulse generating laser system (1),
wherein the control data comprise a group of position control data records, where the deflection mechanism (9) can be actuated by means of one single position control data record, such that a focusing means (4) and the deflection mechanism (9) determine the three-dimensional position of an optical focal point (5) of laser pulses (3) of the laser system (1) within or on the eye lens (6) of a patient's eye (7), depending on the position control data record,
and wherein the group of position control data records is selected such that a diffractive or holographic structure (20) can be generated in the eye lens (6) of a patients' eye (7), if a fluence below the disruption threshold of the material of the eye lens (6) is applied at each focal point (5) by means of at least one ultra-short laser pulse (3).

18. Method according to claim 17, wherein the control data are generated in the laser system (1) itself or are made available to the laser system (1) wirelessly or wire-bound or via an input interface (13) in the form of a file or a data stream.

19. Method according to claim 17, wherein the position control data determine the sequence of a plurality of focal points (5) generated consecutively at different sites.

20. Method according to claim 17, wherein a position control data record fixes two or three space coordinates of a focal point (5).

21. Method according to claim 17, wherein a digital model of the patient's eye (7) to be treated is used for calculating the control data.

22. Method according to claim 17, wherein the control data are adapted to actuate the focusing means (4) and/or the deflection mechanism (9), taking into consideration the optical influence of the transparent components of the patient's eye on the laser pulses, in particular taking into consideration the optical influence of the cornea of the eye.

23. Method according to claim 17, wherein the control data are adapted to actuate the deflection mechanism (9), taking into consideration the optical influence on a laser pulse (3) resulting from the changes in the material or shape of the eye lens (6) by the preceding laser pulses (3).

24. Method according to claim 17, wherein the control data comprise synchronization control data for synchronizing the actuation of the deflection mechanism (9) with the output of laser pulses (3) from an ultra-short pulse laser (2).

25. Method according to claim 17, wherein the position control data are selected such that the diffractive structure (20) that can be generated by the application of the laser pulses (3) is two-dimensional and comprises a plurality of rings (21) or ellipses concentric with respect to each other.

26. Method according to claim 17, wherein the position control data are selected such that the diffractive structure (20) that can be generated by the application of the laser pulses (3) is arranged on an arched or curved surface.

27. Method according to claim 17, wherein the position control data are selected such that the diffractive structure (20) that can be generated by the application of the laser pulses (3) are centered with respect to the optical axis (10) of the patient's eye (7).

28. Computer program with program code for carrying out a method according to claim 17, if the computer program is run on a computer or in the control mechanism (11).

29. Method for treating a patient's eye, wherein a plurality of ultra-short laser pulses is focused at several different focal points on and/or within the natural eye lens of the patient's eye,
wherein a fluence below the disruption threshold of the material of the eye lens is applied at the focal point with a laser pulse, while this fluence is at the same time sufficiently high to cause changes in a material property of the material of the eye lens,
and wherein the position of the focal points is selected such that a diffractive optical structure is generated in the eye lens of the patient's eye by the influence of the focused laser pulses.

30. Method according to claim 29, wherein the diffractive structure is a two- or three-dimensional diffractive structure.

31. Method according to claim 30, wherein the diffractive structure is two-dimensional and comprises a plurality of rings or ellipses concentric with respect to each other.

32. Method according to claim 29, wherein the diffractive structure is arranged on an arched or curved surface.

33. Method according to claim 29, wherein the diffractive structure is centered with respect to the optical axis of the patient's eye.

34. Method according to claim 29, wherein the diffractive structure is shaped such that the eye lens has two or more different focal lengths after treatment.
